(19) United States
(12) Patent Application Publication
(10) Pub. No.: US 2014/0114149 A1
(43) Pub. Date: Apr. 24, 2014

(54) LUNG CANCER DIAGNOSING DEVICE USING PULSE WAVE AND METHOD THEREOF

(71) Applicants: Se-Yun KIM, Seoul (KR); Ji-Hyun JUNG, Seoul (KR); Jae-Hyoung CHO, Seoul (KR)

(72) Inventors: Se-Yun KIM, Seoul (KR); Ji-Hyun JUNG, Seoul (KR); Jae-Hyoung CHO, Seoul (KR)

(73) Assignee: KOREA INSTITUTE OF SCIENCE AND TECHNOLOGY, Seoul (KR)

(21) Appl. No.: 14/043,045

(22) Filed: Oct. 1, 2013

(30) Foreign Application Priority Data
Oct. 19, 2012 (KR) 10-2012-0116839

(51) Int. Cl. A61B 5/07; A61B 5/06; A61B 5/00

(52) U.S. Cl. CPC A61B 5/073; A61B 5/7282; A61B 5/742; A61B 5/7203; A61B 5/065
USPC 600/302

(57) ABSTRACT

A lung cancer diagnosing device and the method thereof are provided. Lung cancer is diagnosed by using pulse waves having a wideband pulse width, so a possibility that a patient is exposed to radiation can be reduced, and since the configuration of transmission and reception circuits and relevant hardware of the device are simple, the size of the overall module is reduced.

In addition, since lung cancer is diagnosed by observing a difference between time delays of pulse waves received by a reception unit according to positions of a transmission module that transmits pulse waves, while moving along digestive organs, a complicated signal processing procedure is not required, and thus, a time required for imaging lung cancer diagnosis results can be reduced.

### CROSS-REFERENCE TO RELATED APPLICATION

Pursuant to 35 U.S.C. §119(a), this application claims the benefit of earlier filing date and right of priority to Korean Application No. 10-2012-0116839, filed on Oct. 19, 2012, the contents of which is incorporated by reference herein in its entirety.

### BACKGROUND

1. Field

The present disclosure relates to a lung cancer diagnosing device using pulse waves and a method thereof.

2. Description of Related Art

Recently, cancer incidence has been steadily increased, and as the importance of the early detection has been growing, various methods for diagnosing a cancer have been proposed. In particular, lung cancer is a malignant tumor originated from the lung, and it is known that deaths from lung cancer account for about 29% of deaths from all the cancers.

Non-invasive medical diagnosis methods for diagnosing cancer include a diagnosis method using X-rays, a diagnosis method using a computed temography (CT), a diagnosis method using continuous waves, and the like.

In the case of the diagnosis method using X-rays, an X-ray light source and a photosensitive plate are disposed and a patient is located therebetween, and images of internal organs of the patient are obtained by using a difference between penetrating power levels of X-rays. Namely, in the diagnosis method using X-rays, a three-dimensional (3D) image of the interior of a human body is expressed as a two-dimensional (2D) film.

Meanwhile, like the diagnosis method using X-rays, the diagnosis method using CT also uses a difference between penetrating power levels of X-rays. In detail, in the case of the diagnosis method using CT, an X-ray light source and a detection device are disposed and a patient is located therebetween. Thereafter, strength and an attenuation constant of X-rays are detected and image processing is performed by a computer to re-configure images of cross-sections of the human body (i.e., the patient). Here, in the case of a cancer diagnosing method using CT, since numerous tomography images are obtained by scanning a human body in a height direction, the patient is exposed to a larger amount of radiation than that of the case based on general X-rays.

Also, in the diagnosis method using continuous waves, lung cancer is diagnosed by measuring an amplitude and a phase of continuous waves, rather than X-rays. However, this method has shortcomings in that a configuration of transmission and reception circuits and relevant hardware is complicated because frequency sweep is required in a frequency band required to be analyzed for diagnosing lung cancer.

### SUMMARY

Therefore, an aspect of the detailed description is to provide a lung cancer diagnosing device using pulse waves capable of reducing an amount of radiation exposed to a patient and simplifying circuits of a transmission and reception device or a hardware configuration in relation to the transmission and reception device included in the lung cancer diagnosing device, thus reducing a size of the overall module corresponding to the lung cancer diagnosing device, and a lung cancer diagnosing method.

Another aspect of the detailed description is to provide a lung cancer diagnosing device which uses pulse waves resistant to a signal-to-noise ratio and measures a delay time during which pulse waves transmitted from a transmitter arrives at a receiver to diagnose lung cancer, whereby lung cancer is diagnosed without having to perform a complicated signal processing procedure, and a lung cancer diagnosing method.

To achieve these and other advantages and in accordance with the purpose of this specification, as embodied and broadly described herein, a lung cancer diagnosing device includes: a reception unit configured to receive a pulse wave propagated from a transmission module moving along digestive organs after being taken through an oral cavity; a time delay measuring unit configured to measure time delay information from the received pulse wave; and a controller configured to diagnose lung cancer on the basis of time delay information according to a position of the transmission module.

In an embodiment of the present invention, the controller may compare the measured time delay information with a predetermined reference time delay range, and when there is a time delay in a particular position of the transmission module according to the comparison results, the controller may diagnose lung cancer.

In an embodiment of the present invention, the transmission module may include: a pulse wave generating unit configured to generate a pulse wave by using a clock signal; a transmission antenna configured to transmit the generated pulse wave to the reception unit; and a communication unit configured to communicate with the reception unit through a fixed line or wirelessly.

In an embodiment of the present invention, the reception unit may be positioned in proximity of the outside of a human body and receive a pulse wave propagated from the transmission module through an internal antenna.

In an embodiment of the present invention, the reception unit may include a low noise amplifier for reducing noise of the received pulse wave.

In an embodiment of the present invention, the lung cancer diagnosing device may further include a communication unit configured to transmit a pulse wave transmission request signal to the transmission module and receive information regarding a position of the transmission module from the transmission module.

In an embodiment of the present invention, the time delay measuring unit may measure time delay information of the received pulse wave by using a time delay difference between the received pulse wave signal and the pulse wave signal transmitted by the transmission module, and transmit the measured time delay information to the controller.

In an embodiment of the present invention, the transmission module and the reception unit may transmit and receive signals by using a wideband signal communication scheme.

In an embodiment of the present invention, the lung cancer diagnosing device may further include: a storage unit configured to store the measured time delay information.

In an embodiment of the present invention, the lung cancer diagnosing device may further include: a display unit configured to display the measured time delay information and the diagnosis results from the controller according to a control command from the controller.

To achieve these and other advantages and in accordance with the purpose of this specification, as embodied and broadly described herein, a lung cancer diagnosing method using a pulse wave through a transmission module taken into a body through an oral cavity and a reception unit positioned in proximity of outside of the body and communicating with the transmission module, includes: transmission a pulse wave by the transmission module moving along digestive organs after being taken through an oral cavity; receiving, by the reception unit, the pulse wave propagated from the transmission module; measuring time delay information from the received pulse wave; and diagnosing lung cancer on the basis of the time delay information according to a position of the transmission module.

In an embodiment of the present invention, in the measuring of the time delay information, a time delay difference between the received pulse wave signal and the pulse wave signal transmitted by the transmission module may be measured.

In an embodiment of the present invention, the diagnosing of lung cancer may include: comparing the measured time delay information with a predetermined reference time delay range; and when there is a time delay at a particular position of the transmission module according to the comparison results, diagnosing lung cancer.

In the case of the lung cancer diagnosing device and the method thereof according to embodiments of the present invention, since lung cancer is diagnosed by using pulse waves having a wideband pulse width, a possibility that a patient is exposed to radiation can be reduced, and since the configuration of transmission and reception circuits and relevant hardware of the device are simple, the size of the overall module is reduced.

In addition, In the case of the lung cancer diagnosing device and the method thereof according to embodiments of the present invention, since lung cancer is diagnosed by observing a difference between time delays of pulse waves received by a reception unit according to positions of a transmission module that transmits pulse waves, while moving along digestive organs, a complicated signal processing procedure is not required, and thus, a time required for imaging lung cancer diagnosis results can be reduced

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE DRAWINGS

The accompanying drawings, which are included to provide a further understanding of the invention and are incorporated in and constitute a part of this specification, illustrate exemplary embodiments and together with the description serve to explain the principles of the invention.

In the drawings:

FIG. 1 is a view illustrating the use of a lung cancer diagnosing device according to an embodiment of the present invention;

FIG. 2 is a schematic block diagram illustrating a detailed configuration of the lung cancer diagnosing device according to an embodiment of the present invention;

FIGS. 3 and 4 are flow charts illustrating a process of a lung cancer diagnosing method according to an embodiment of the present invention, respectively; and

FIG. 5 is a graph showing a time delay of a pulse wave received when lung cancer is discovered according to an embodiment of the present invention.

### DETAILED DESCRIPTION

Description will now be given in detail of the exemplary embodiments, with reference to the accompanying drawings. For the sake of brief description with reference to the drawings, the same or equivalent components will be provided with the same reference numbers, and description thereof will not be repeated.

The terminology used herein is for the purpose of describing particular embodiments only and is not intended to be limiting of the invention. Unless otherwise defined, all terms used herein have the same meaning as commonly understood by one of ordinary skill in the art to which this invention pertains, and should not be interpreted as having an excessively comprehensive meaning nor as having an excessively contracted meaning.

It will be further understood that terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

A lung cancer diagnosing device using a pulse wave and a method thereof according to embodiments of the present invention will be described with reference to the accompanying drawings.

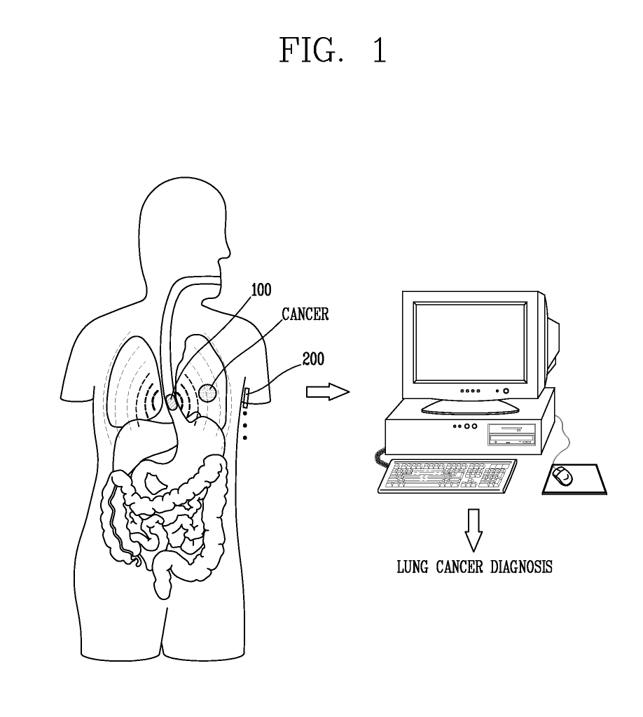

FIG. 1 is a view illustrating the use of a lung cancer diagnosing device according to an embodiment of the present invention. As illustrated in FIG. 1, the lung cancer diagnosing device according to an embodiment of the present invention includes a pulse wave transmission module 100 and a pulse wave reception module 200.

The pulse wave transmission module 100 and the pulse wave reception module 200 may be connected wirelessly or by a fixed line. In the case of wireless connection, the pulse wave transmission module 100 may be configured as a capsule and taken through the oral cavity of a body as an inspection target (e.g., a patient).

A magnitude of a pulse wave signal generated by the pulse wave transmission module 100 may be regulated and transmitted through an external device (not shown). Also, a position of the pulse wave transmission module 100 within the body may be regulated.

The pulse wave transmission module 100 may be taken through the oral cavity and move in a vertical direction along digestive organs. Also, the pulse wave transmission module 100, while moving in the vertical direction, may transmit pulse waves to the pulse wave reception module 200 positioned outside of the body.

Meanwhile, the pulse wave reception module 200 may receive the pulse waves transmitted from the pulse wave transmission module 100, and measures a delay time from the received pulse waves. The measured delay time may be checked by the user through a monitoring screen.

The pulse wave reception module 200 diagnoses lung cancer on the basis of the measured delay time according to a position of the pulse wave transmission module 100. In order to diagnose lung cancer, preferably, the pulse wave reception module 200 is installed or positioned to be distributed wider than a range surrounding the lung in the body.

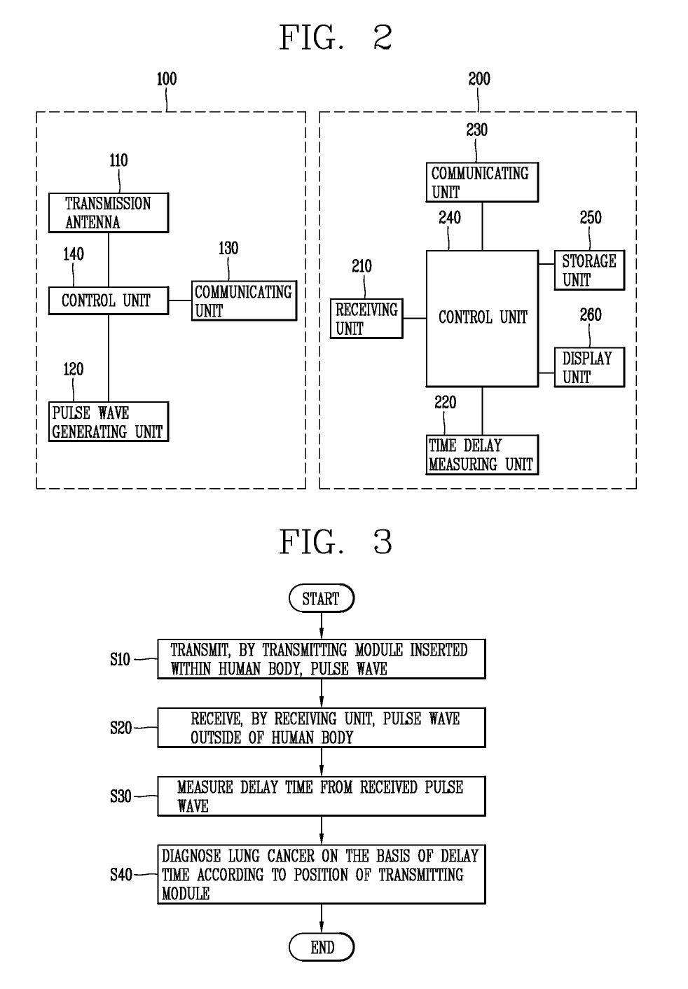

FIG. 2 is a schematic block diagram illustrating a detailed configuration of the lung cancer diagnosing device according to an embodiment of the present invention.

As illustrated in FIG. 2, the lung cancer diagnosing device includes the pulse wave transmission module 100 configured to transmit a pulse wave and the pulse wave reception module 200 configured to receive a pulse wave. In detail, the pulse wave transmission module 100 includes a transmission antenna 110, a pulse wave generating unit 120, a communication unit 130, and a control unit 140. The pulse wave reception module 200 includes a reception unit 210, a time delay measuring unit 220, a communication unit 230, a control unit 240, a storage unit 250, and a display unit 260.

Hereinafter, a configuration of the pulse wave reception module 200 of the lung cancer diagnosing device will be described in detail.

The reception unit 210 receives pulse waves transmitted from the pulse wave transmission module 100 moving along the digestive organs after being taken through the oral cavity. Namely, the reception unit 210 is positioned in the proximity of the body and receives pulse waves propagated from the pulse wave transmission module 100 through an internal antenna thereof.

The reception unit 210 may include a low noise amplifier (LNA) in order to reduce noise of pulse waves received from the power pulse transmission module 100.

Also, the reception unit 210 may transmit and receive signals to and from the pulse wave transmission module 100 through a wideband signal communication scheme. For example, an ultra-wide band (UWB) signal communication scheme in which pulse waves using a frequency band of a few GHz or higher in a baseband are transmitted and received may be employed.

The time delay measuring unit 220 measures a delay time from the received pulse waves. In detail, the time delay measuring unit 220 measures a delay time of the pulse waves by using a time delay difference between the pulse wave signal and the pulse wave signal transmitted by the pulse wave transmission module 100. The measured delay time may be transmitted to the control unit 240 and used to diagnose lung cancer.

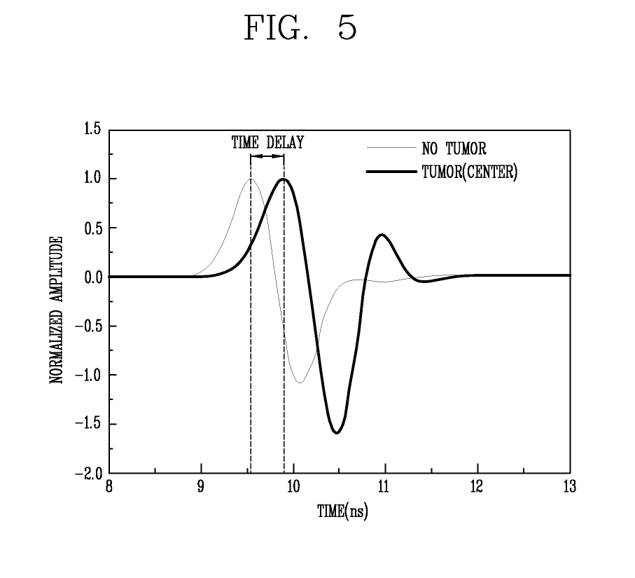

In this connection, FIG. 5 is a graph showing a time delay of received pulse waves when lung cancer is discovered according to an embodiment of the present invention. As shown, when lung cancer exists, pulse waves transmitted from the pulse wave transmission module 100 are delayed to reach the reception unit 210. Here, a waveform of the received pulse wave may vary according to the transmitted pulse wave, but a process of determining whether lung cancer exists by measuring a delay time is the same.

The time delay measuring unit 220 may be implemented as a clock correlator (not shown) that may be able to capture (or check) synchronization of a received pulsed signal (or a pulse wave) to thereby measure an arrival delay time of the pulse signal transmitted from the transmitter to the reception unit 210.

When a pulse is received as a part of a stream of pulses transmitted at a predetermined time interval from the transmitter, the reception unit 210 of the reception module 200 may be able to detect (or determine) an order of the pulse in the steam of pulses.

The pulse wave signals transmitted at predetermined time intervals from the pulse wave transmission module 100 may be stored as template signals in the clock correlator.

Also, when a pulse wave signal received by the reception unit 210 of the reception module 200 is applied as a newly input signal to the clock correlator and when previously input signals (or signals stored in the form of template signals) and newly input signals are identical or similarly identical, the clock correlator may generate a signal indicating that they are identical and transfer the same to the controller 240.

Whether the previously input signals and the newly input signals are identical or similar may be determined according to various methods. For example, the time delay measuring unit 220 may determine whether a template signal corresponding to any one of the previously input signals and the newly input signal are identical or similar on the basis of correlation between the template signal and the newly input signal. To this end, the time delay measuring unit 220 may calculate the correlation on the basis of a correlation function. Besides, it will be obvious to a person skilled in the art that the sameness or similarity is determined according to various other methods.

According to a modification, the sameness or similarity between the previously input signals and the newly input signal may be determined by the controller 240.

On the basis of the signal determined to be identical, the controller 240 may determine whether the newly input signal is identical to which one of the previously input signals or not identical.

On the basis of the determination, the controller 240 may observe whether a delay time of the predetermined newly input signal exceeds a reference time delay range, to diagnose lung cancer.

Namely, the controller 240 may observe whether a pulse wave signal received by the reception unit 210 of the reception module 200 is delayed by more than a particular period of time at a particular point in time to measure delay time information, and when the observed delay time exceeds the predetermined reference time delay range, the controller 240 may determine lung cancer.

FIG. 5 shows comparison results between the waveform of the pulse signal transmitted from the transmission module 100 and that of the pulse signal received by the reception unit 210 of the reception module 200. In FIG. 5, it can be seen that there is a time delay difference of about 0.5 ns at a point 9.5 ns.

Here, the size of the difference in the delay time may vary according to a shape, a size, and the like, of lung cancer.

The control unit 240 may control a general operation of the lung cancer diagnosing device, and diagnose lung cancer on the basis of measured delay time according to a position of the pulse wave transmission module 100.

In detail, the control unit 240 compares the measured delay time with a predetermined reference time delay range. According to the comparison results, when it is checked that there is a time delay at a particular position of the pulse wave transmission module 100, the control unit 240 diagnoses lung cancer. The control unit 240 may check whether there is a time delay by, for example, analyzing a pulse wave graph displayed on a monitoring screen. To this end, a microcomputer, a central processing unit, or the like, having functions such as comparison, determination, and the like, may be used as the control unit 240.

Also, the control unit 240 may calculate a rough size or distribution degree of lung cancer by using measured delay time.

The communication unit 230 may transmit a signal for requesting transmission of a pulse wave from the transmission module 100 and/or receive information regarding a position of the pulse wave transmission module 100 from the pulse wave transmission module 100.

The storage unit 250 stores the delay time measured by the time delay measuring unit 220. To this end, the storage unit 250 may be configured as a general ROM, RAM, a flash ROM, an SD card, or the like, and the storage unit 250 may further store the diagnosis results from the control unit 240.

The display unit 260 may display the delay time measured by the time delay measuring unit 220 and the lung cancer diagnosis results performed by the controller 240. Displaying of the delay time and the lung cancer diagnosis results on the display unit 260 may be performed according to a control command of the controller 240.

Hereinafter, a configuration of the pulse wave transmission module 100 of the lung cancer diagnosing device will be described in detail.

The pulse wave generation unit 120 generates a pulse wave by using a clock signal. The transmission antenna 110 transmits the pulse wave generated by the pulse wave generation unit 120 to the reception unit 210 of the reception module 200. The communication unit 130 communicates with the reception unit 210 of the pulse wave reception module 200 through a fixed line or wirelessly.

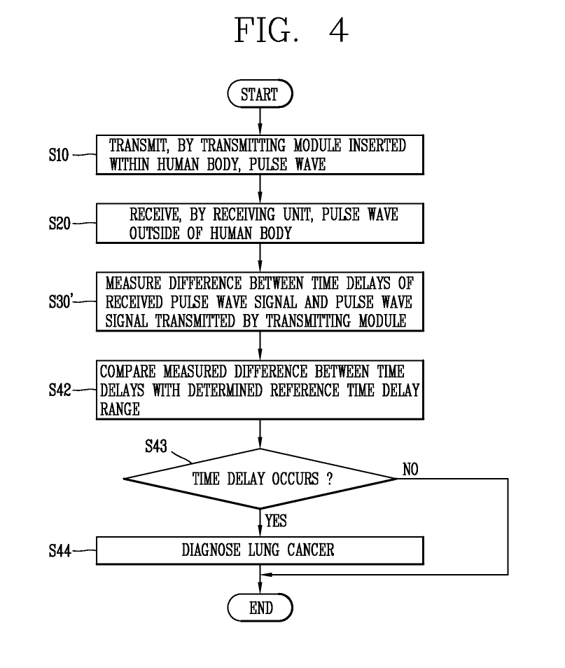

FIGS. 3 and 4 are flow charts illustrating a process of a lung cancer diagnosing method according to an embodiment of the present invention, respectively. The method for diagnosing lung cancer may be performed by the lung cancer diagnosing device including the pulse wave transmission module 100 taken in a body through an oral cavity and a reception unit 210 positioned to be adjacent to a body and communicating with the pulse wave transmission module 100.

First, referring to FIG. 3, the pulse wave transmission module 100 is taken through an oral cavity, and transmits a pulse wave, while vertically moving along the digestive organs (S10). Then, the reception unit 210 of the pulse wave reception module 200 positioned outside of the body receives the pulse wave propagated from the transmission module 100 (S20). The reception module 200 measures the delay time from the received pulse wave (S30). Whether there is lung cancer is diagnosed on the basis of measured delay time according to a position of the transmission module 100 (S40).

FIG. 4 specifically illustrates a process of diagnosing lung cancer by using time delay information.

The transmission module 100 may be taken through the oral cavity of a target human body.

The taken transmission module 100, vertically moving along the digestive organs, may transmit a pulse wave (S10).

The reception unit 210 of the pulse wave reception module 200 may receive the pulse wave propagated from the transmission module 100 (S20).

The reception module 200 may observe a time delay of the received pulse signal and the pulse signal transmitted from the transmission module 100 to measure time delay information (S30′).

Thereafter, the pulse wave reception module 200 may compare the measured time delay information with a reference time delay range (S42).

The pulse wave reception module 200 may determine whether there is a time delay in the received pulse signal on the basis of the comparison results (S43).

When there is a time delay according to the determination results, lung cancer may be diagnosed (S44).

As described above, in the case of the lung cancer diagnosing device and lung cancer diagnosing method according to embodiments of the present invention, since lung cancer is diagnosed by using a pulse wave having a wideband pulse width, a possibility that a patient is exposed to radiation is reduced, and since the configuration of the transmission and reception circuits and associated hardware of the device is simplified, a size of the overall module is reduced. Also, since lung cancer is diagnosed by observing a time delay difference in received pulse waves according to a transmission position of the pulse wave transmission module, a complicated signal processing procedure is not necessary and a time required for imaging lung cancer diagnosis results can be shortened.

The foregoing embodiments and advantages are merely exemplary and are not to be considered as limiting the present disclosure. The present teachings can be readily applied to other types of apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds are therefore intended to be embraced by the appended claims.

What is claimed is:

1. A lung cancer diagnosing device comprising:
a reception unit configured to receive a pulse wave propagated from a transmission module moving along digestive organs after being taken through an oral cavity;
a time delay measuring unit configured to measure time delay information from the received pulse wave; and
a controller configured to diagnose lung cancer on the basis of time delay to information according to a position of the transmission module.

2. The lung cancer diagnosing device of claim 1, wherein the controller compares the measured time delay information with a predetermined reference time delay range, and when there is a time delay in a particular position of the transmission module according to the comparison results, the controller diagnoses lung cancer.

3. The lung cancer diagnosing device of claim 1, wherein the transmission module comprises:
a pulse wave generating unit configured to generate a pulse wave by using a clock signal;
a transmission antenna configured to transmit the generated pulse wave to the reception unit; and
a communication unit configured to communicate with the reception unit through a fixed line or wirelessly.

4. The lung cancer diagnosing device of claim 1, wherein the reception unit is positioned in proximity of the outside of a human body and receive a pulse wave propagated from the transmission module through an internal antenna.

5. The lung cancer diagnosing device of claim 4, wherein the reception unit includes a low noise amplifier for reducing noise of the received pulse wave.

6. The lung cancer diagnosing device of claim 1, further comprising:
a communication unit configured to transmit a pulse wave transmission request signal to the transmission module and receive information regarding a position of the transmission module from the transmission module.

7. The lung cancer diagnosing device of claim 1, wherein the time delay measuring unit measures time delay information of the received pulse wave by using a time delay difference between the received pulse wave signal and the pulse wave signal transmitted by the transmission module, and transmits the measured time delay information to the controller.

8. The lung cancer diagnosing device of claim 1, wherein the transmission module and the reception unit transmit and receive signals by using a wideband signal communication scheme.

9. The lung cancer diagnosing device of claim 1, further comprising:
a storage unit configured to store the measured time delay information.

10. The lung cancer diagnosing device of claim 1, further comprising:
a display unit configured to display the measured time delay information and the diagnosis results from the controller according to a control command from the controller.

11. A lung cancer diagnosing method using a pulse wave through a transmission module taken into a body through an oral cavity and a reception unit positioned in proximity of outside of the body and communicating with the to transmission module, the method comprising:
transmitting a pulse wave by the transmission module moving along digestive organs after being taken through an oral cavity;
receiving, by the reception unit, the pulse wave propagated from the transmission module;
measuring time delay information from the received pulse wave; and
diagnosing lung cancer on the basis of the time delay information according to a position of the transmission module.

12. The method of claim 11, wherein in the measuring of the time delay information, a time delay difference between the received pulse wave signal and the pulse wave signal transmitted by the transmission module is measured.

13. The method of claim 11, wherein the diagnosing of lung cancer comprises:
comparing the measured time delay information with a predetermined reference time delay range; and
when there is a time delay at a particular position of the transmission module according to the comparison results, diagnosing lung cancer.
